(19) United States
(12) Patent Application Publication
(10) Pub. No.: US 2023/0120861 A1
(43) Pub. Date: Apr. 20, 2023

(54) MULTIDIMENTIONAL HEALTHCARE OUTCOME PREDICTOR

(71) Applicant: Medicardia Health, Inc., Mequon, WI (US)

(72) Inventors: Indrajit Choudhuri, Mequon, WI (US); David Geddam, Ladue, MO (US); Kiran Karunakaran, Pleasanton, CA (US)

(21) Appl. No.: 17/968,624

(22) Filed: Oct. 18, 2022

Related U.S. Application Data
(60) Provisional application No. 63/257,063, filed on Oct. 18, 2021.

(51) Int. Cl. G16H 50/20; G16H 15/00; G16H 70/20; G06F 40/40

(52) U.S. Cl. CPC G16H 50/20; G16H 15/00; G16H 70/20; G06F 40/40

(57) ABSTRACT

A system for medical data management for a computing device providing indications for patient care, the system executes instructions for managing data resources associated with patient care including data extraction and for identifying an element of patient data received, which may be based on clinical medical data associated with a patient. The system determines a first output associated with medical data. Via the clinical intelligence engine, the system uses prediction to determine element values and updated outputs of medical data related to predicted patient health care plans and health and creates a visualization graphical output containing areas that are updated in the second output for improved health care planning and visualization of medical data.

### CROSS REFERENCE TO RELATED APPLICATIONS

This application claims the benefit of U.S. Provisional Pat. Appl. No. 63/257,063, filed Oct. 18, 2021. This application is herein incorporated by reference in its entirety.

### BACKGROUND

Currently health care providers keep records of patients in various folders and in various software programs. The amount of time that a provider needs to go in and out of files, folders, and programs is time consuming and data about a patient can be missed. The many folders and files that are access to assemble patient information can often be time consuming and information in the records can be missed. One record may have the patient's health history and another file may have upcoming or ordered diagnostic testing information. Medical records are often one or two dimensional and are viewed as a graphical output of data linearly that shows a timeline of progress and curves of the patient statistics. Clinicians have to review different folders and access different records and view linear output graphics of patient data in order to analyze the data for progress. Looking in various charts and records can often cause much delay and not present the full scope of the patient's health.

### BRIEF DESCRIPTION

The disclosure is for an application that provides electronic database records in a graphical format more efficient and useful to a healthcare provider, but it also provides medical information about the patient to improve the likelihood of success determination. Already out there are electronic health systems, health records, which are digitized versions of patient's medical documents, whether they are notes, diagnostic test results, communications, laboratory information, demographic information, etc. The information is stored in a database that is not organized in a manner that a medical professional would use the information. When a physician accesses a patient health record, there are a variety of folders, with separate groupings of information, that contain all the various types of results for that patient. Non-limiting examples, notes, bloodwork, diagnostic test results and other medical information stored. Other separate folders that contain information about upcoming events, such as appointments, orders for blood work, or orders for testing. To use information, the clinician needs to go into each one to see if there is updated information since the patient was last seen by that particular provider.

For continuity, the clinician may pick up where the provider last left off. There may be a time gap between appointments and the clinician may fill the time gap longitudinally with what happened from the last visit to the current visit, adding information acquired during the check up from many sources such as diagnostics, interviewing the patient, checking the patient, etc. and learning what is new or changed since the patient's last visit. A clinician may use all of that information from what's transpired since the last visit to determine next appropriate health care planning and patient care management steps.

### SUMMARY OF THE INVENTION

A system for medical data management for a computing device providing indications for patient care, the system comprising a memory and at least one processor for processing data. The system has different embodiments and advance health care predictive health plan and treatment of certain patients. The processor can execute instructions for managing data resources associated with patient care including data extraction and for identifying an element of patient data received. The processors may receive data, based on clinical medical data associated with a patient and may determine a first output associated the received clinical medical data. The first output has at least one area of data associated with the health of the patient.

The system may determine, based on a set of rules of a clinical intelligence engine for providing patient care, a weighted value for each of the respective elements of the received clinical medical data determines, wherein the weighted value is based on a likelihood of a clinician using the data for determining patient care. The system may further determine, based on the weighted value of each of the respective elements of the received clinical medical data, as association with the at least one area of the first output. In some embodiments, the system may select, based on the determined weighted value of each of the respective elements in relation to a threshold and the association with the at least one area of the first output, the element of the clinical medical data, and in some instances, determine, based on the determined association of the at least one area, a second output, wherein the at least one of data associated with the health of the patient is included in the second output indicating a suggestion for patient care that the clinician would likely use for patient care. The threshold is based on a predetermined value, and when the determined value of the element of the clinical medical data is above the predetermined threshold value, then the element is selected as likely used by the clinician as part of the patient care plan. The first output may also apply a prediction and clinical intelligence engine to determine the first output. In some embodiments, the identifying the element is based on discretization, of the element of patient data received. At a graphical user interface, the second output with the likely suggestion for patient care, wherein the second output incorporates the elements associated with clinical medical data, in the at least one area of data. The second output may include the selected element in the second output to a provider taking steps suggested for patient care. In some embodiments, the data extraction is based on natural language processing. The medical data system of claim 1, wherein the system identifies natural language in the clinical medical data to determine the second output.

In some embodiments, a clock may be applied for tracking the received clinical medical data and for a specific clinician for accessing the medical data system. The clock may be used to track data as well as determining values of the data and amount of time spent on providing patient health care. The clinical medical data comprises, but is not limited to: vital signs, notes, physician input, health, tests, images documents, files, graphical data, clinician input, test results, measurements, documents, photographs, scans, echocardiogram, x-rays, images, and health information associated with the patient.

In some instances, a value for received a first output based on the received data received from the clinical intelligence engine, which applies rules to the received data at least from the rules engine, and the managed data associated with patient care including care notes, monitoring thresholds and parameters. The managed data associated with patient care includes clinician notes, patient medical monitoring, thresholds, care guidelines, tests, vitals, and parameters. The clinician may input data into the medical data system and is part of the determination of the first output, the second output, or both the first and second outputs.

In some embodiments, a rules engine of the clinical intelligence engine, may be used for at least one of data verification elements such as the source of information, pattern of information and a likelihood of reliability of data, a clinician may use as part of providing health care or a treatment plan. The second output may then include the reliable information and include a graphical display of data representing a visual display of suggested for patient care. The second output includes an evaluation based on a clinician inference. In some instances, data determined through the rules engine is determined through a rule or a set of rules in the clinical intelligence platform, and determines an inference or outcome based on the application of the rule or the set of rules. In some instances, the second output is based on at least one of clinical guidelines, AHA or ACC guidelines, Federal or State medical and healthcare guidelines, or any combination thereof.

The executed instruction for managing data resources may integrate with the at least one processor and is in response to at least one of receiving data, data management, and a combination thereof. the received data from the rules engine includes a verification of data where the likelihood of meeting patient care guidelines and using the data in patient care.

The executed instruction for managing data resources integrates with the at least one processor for determining if the received data, including at least one of but not limited to health care data, lab result data, testing data, monitoring data, vital sign associated data, medical device data, clinical data, and healthcare data, and the received data is likely associated to the area of the first or second output. The output that includes an integration with medical data and may be associated with a patient chart or medical history.

In some embodiments, the processor may execute storing data in the memory including, but not limited to, storing patient chart and long records. For example, overall set up information related to patient chart, vital signs, images, pdfs, documents, clinician input, device data received, may be stored. The processor may also execute storing data in the memory including, but not limited to, storing selected elements of the received clinical medical data in association with the determined weighted value. The rules engine may determine, based on executed instruction for at least managing data resources, the output that includes an integration with medical data associated with a patient chart. The determined second output may be based on the likelihood of improved or known treatment care guideline for a given patient based on the received clinical intelligence engine data associated with the patient.

The received provider input is based on input an integrated workflow from the at least one provider, wherein the provider input has an authentication and a role, based on an access level, which allows the provider with a certain allowable set of functionalities related to addressing the patient care. In some embodiments, the at least one provider communication interface may be with a camera interconnected with the system and graphical user interface of the computing device.

In some embodiments, the second output may include a radar graph that includes an indication of each condition. The radar incorporates each condition's validated risk predictors and then graphically represents how the result may impact the heart score or overall health of the patient. In some instances, a RAF score may indicate a high risk factor the likelihood of success of care. In other embodiments, the processors may determine cost and analyze the cost to the patient and health care provider and running an evaluation of the predicted outcome of care. In some embodiments, the second output is further based on context-based determinations or indication mapping, diagnostic conditions or codes, or chronic condition criteria, based as well on related diagnostic codes (e.g., ICD) or specific keywords related to clinical guidelines or therapies discussed in the guidelines.

The second output may contain a predictive model to show medical conditions, wherein the second output contains at least one anatomical model, curve, radar plot, data plot, diagram, chart, schematic, graph, graphical model, or any combination thereof. The second output may be searchable based on a set of parameters to allow search and used in the different engine. In some embodiments, some steps that may be executed may include mapping, billing, searching, saving in a database with associations, saving standards and codes that is associated with data in a network server with data, and other tools may be applied.

The above summary is not intended to describe each disclosed embodiment of every implementation of the present disclosure. The brief description of the drawings and the detailed description which follows more particularly exemplify illustrative embodiments. In the drawings, the layers are not necessarily drawn to scale. In this disclosure a single item may include the plurality, or more than one, of the item. References to plurals, may also include a single of the same item.

### BRIEF DESCRIPTION OF THE DRAWINGS

FIG. 1 is an example illustration of a system for managing medical data, in accordance with one or more aspects of the present disclosure.

FIG. 2 is an example illustration of a medical data dashboard associate with a patient and the patient's profile history and medical condition and treatment within a dashboard, in accordance with one or more aspects of the present disclosure.

FIG. 3 is a flow diagram through the clinical intelligence engine and application of using NLP (Natural Language Processing) dataflow diagram, in accordance with one or more aspects of the present disclosure.

FIG. 4 is an example flow diagram of some of the data processed and part of the health care plan prediction second output such as the radar and health score, in accordance with one or more aspects of the present disclosure.

FIG. 5 is an example of a data lake eco system that shows some of the data that goes into the patient analysis and precision health care including the processing of the data, in accordance with one or more aspects of the present disclosure.

FIG. 6 is an example process diagram of the dashboard interface, in accordance with one or more aspects of the present disclosure.

FIG. 7a is an example of the radar chart of patient medical data, in accordance with one or more aspects of the present disclosure.

FIG. 7b is an example of the display during a video conference with a clinician, in accordance with one or more aspects of the present disclosure.

FIG. 7c is an example of data accessible relating to medical history and accessing records and care plans associated with a patient, in accordance with one or more aspects of the present disclosure.

FIG. 7d is an example of the display related to patient medical data and patient monitoring providing a risk assessment, in accordance with one or more aspects of the present disclosure.

FIG. 8 is an example illustration of types of diagrams that the application may analyze, in accordance with one or more aspects of the present disclosure.

FIG. 9 is an example system diagram of the application, in accordance with one or more aspects of the present disclosure.

### DETAILED DESCRIPTION

Various customer interfaces and embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims. It is understood that various omissions and substitutions of equivalents are contemplated as circumstances may suggest or render expedient, but these are intended to cover applications or embodiments without departing from the spirit or scope of the claims attached hereto. Also, it is to be understood that the phraseology and terminology used herein are for the purpose of description and should not be regarded as limiting.

The application is an interface between all of these databases, to automatically extract info and visualize it for a clinician within one pane of glass or one view. Instead of the tedious tasks of having to open up the last note, look in another section for medical and surgical history, open up another folder for vital signs, and then open yet another folder to find out the results of the last echo or catheterization or other, all of the aforementioned (and more) data is visualized in one screen. With one screen dashboard visual, the med provider can use to actively provide care to the patient.

To create the dashboard, there are many components. Certain terms are used herein that have the following non-limiting meanings:
Dashboard: Radar Plot combined with the Heart Score and risk assessment basis (in one embodiment the Framingham Risk Calculator based risk assessment, but others are possible).
Heart Chart: is a radar plot with risk variables of Heart Diseases, Habits, Vitals, Biomarkers, ECG, Imaging, and Comorbidities.
Heart Score/Health Score: is a value or an indication that represents the overall cardiovascular health of an individual. In general, Heart Scores may have a range. The range in one nonlimiting embodiment may be from 300 to 1000, with lower scores generally indicating greater number or severity of disease or risk factors, or cardiovascular abnormalities, and higher scores indicating fewer number or severity of disease or risk factors, or cardiovascular abnormalities. Larger or smaller ranges are possible.

The improvement of the system over standard data displays is to show information all in one display, it allows an understanding of the important information is one graphical representation. It may also indicate what the current status of the patient which may provide valuable information for next steps in continuing treating the patient or may impact the patient's health. The dashboard of this disclosure includes medical information about the patient and information that may provide information about resulting outcomes if treatment or action of the patient is complied with. The application sifts through the many medical records, analyses the applications and relevancy, saving the clinician time and reducing the risk of missing valuable data. Every piece of info has to be searched for providing care, which creates risk that information is not seen or reviewed. The application reduces this risk, basing the patient's health dashboard on information. Additionally, the dashboard may improve the graphical imaging and focus of the information by showing the clinician what they need to see. The dashboard may not show data about the patient or the patient's health that may not be needed or irrelevant to the current health of the patient at a particular moment. The application displays a lot of data that may be relevant information in a split second that is easy to view in the dashboard, so the clinician doesn't have to waste any valuable time looking for it.

In some embodiments, the application may be executed on a device and the clinician may login through an account or accessing medical. To maintain privacy of patients, medical records may require a login or form of authentication. It may be a simple process or a multi-step process for accessing patient data. Different authentication techniques explained ahead may be used for accessing medical data. These techniques may in some embodiments include Single Sign On (SSO), which allows the use a third-party service such as AWS SSO or Basic Authentication which is managed by the Admin Panel where the users are associated with a different group such as Administrators, Service, Physicians or Nurse Practitioners who play different roles in the system. In another example, the system is integrated with an existing network records management system and uses the system's authentication. The user may be required to setup their own password due to HIPAA compliance requirements for Authentication. Additionally, all the changes to a given patient chart are monitored and recorded based on who reviewed or edited the patient charts. The system is also completely protected by a firewall and using the latest cybersecurity transfer encryption to ensure all key transactional elements of the patient data flow are kept secure.

In some embodiments, the user or a clinician may first log into their instance of the platform that may then show their list of patients that are going to be seen. In another embodiment, the user could search for a particular patient from a plurality, even if not related to an appointment. When the chart is opened, the system auto will receive a set number of pieces of inform (vitals, notes, demographics, diagnostic test results, medical procedures, vitals, health statistics, observations, etc.) to populate the dashboard, and it does that immediately, so that the auto chart prep is an immediate time savings and shows the clinician in one view, everything they need to take care of the patient. In other embodiments, the data is presented in a dashboard that includes organized areas of medical data. In one embodiment, a radar plat may be part of the display showing cardiac information. In another area, a health score may be visible related to the overall health of the patient. In another area, medical conditions may be displayed related to the medical record or indicated medical conditions. The display may also include interactive icons that the clinician or patient may access to take actions, such as schedule appointment, past/present/future visit information, location, patient account information, clinician account information, diagnostic information, demographic information, change in health, change in medical diagnostic information, and other medical or account related data may be part of the graphical display.

The graphical output may be based on multiple parameters of the patient's health. The parameters of data in the medical and health graphical display areas are based on age, weight, body mass index (BMI), habits that may affect the patient's health such as smoking/vaping/alcohol/drugs/etc., homocysteine, hemoglobin A1C (HGBA1C), glucose level, total cholesterol (Tchol), high density lipoprotein (HDL), erythrocyte sedimentation rate (ESR), high sensitivity C-reactive protein (hs-CRP), QRSDURAT, troponin (TROP), Systolic Blood Pressure SBP, Diastolic Blood Pressure DBP, forced vital capacity (FVC), BNP, Electrical Impulse QRS Duration QRSDURAT, Electrical Impulse QTC Interval QTCINTERV, Electrical Impulse QTC Short Duration QTCSHORT, calcium score (CALCSCORE), Ejection Fraction EF, LVTHCKNS, LVH, CXR, HNTNRx, OSA, chronic obstructive pulmonary disease (COPD), CAROTIDDZ, AORTICDZ, CORRVSC, aortic valve (AOVALV), mitral valve (MITRVALV), DM, CAD, SCD, MI, CHF, AF, PAD, STROKEHEM, TIA/STROKEISCH, and other medical related data that indicate the health status or disease of the patient. The parameters may include information about procedures or medical events occurring in the past.

Various Risk Factors and Definitions may be as follows and used in the system:

| Term | Definition |
|---|---|
| HGBA1C | Hemoglobin A1C Measurement |
| A1C | Glucose Level |
| Tchol | Total Cholestrol |
| HDL | High Density Lipids |
| ESR | Erithrocyte Sedimentation Rate |
| Hs-CRP | High Sensitivity C-Reactive Protein |
| QRSDUR | QRS Duration |
| Trop | Troponin |
| SBP | Systolic Blood Pressure |
| DBP | Diastolic Blood Pressure |
| QRSDURAT | QRS Duration |
| QTCINTERV | QTC Interval |
| QTCSHORT | QTC Short Interval |
| CALCSCORE | Coronary CT calcium score |
| LVTHCKNS | LV wall thickness |
| LVH | LV Hypertrophy |
| CXR | Chest Radiograph |
| HNTNRx | High Sensitivity Troponin Prescription |
| OSA | Obstructive Sleep Apnea |
| (COPD) | chronic obstructive pulmonary disease |
| CAROTIDDZ | Carotid Artery Disease |
| AORTICDZ | Aortic Valve Disease |
| CORRVSC | Coronary Vascularization |
| AOVALV | aortic valve |
| MITRVALV | mitral valve |
| DM | Diabetic mellitus |
| CAD | Coronary Artery Disease |
| SCD | Sudden Cardiac Disease |
| MI | Myocardial Infarction |
| CHF | Congenital Heart Failure |
| AF | Atrial Fibrillation |
| PAD | Peripheral Artery Disease |
| STROKEHEM | Hemorrhagic Strokes |
| TIA/STROKEISCH | Trans Ischemic Attack |

These are conditions and terms that are used patient care and have particular weighted levels of concern that a clinician may look at and may consider when determining the treatment plan consideration. These elements in any document or received data would be weighted and applied and given a value. There may be other data received from med records system, real time note entry, received from wearables and home medical devices and implanted defibrillator and pacemakers. In order to visualize data or graphically display the information, the application may then determine if various features immediately become prominent or if identification of health information is identified, becoming part of the graphical display for the clinician. To determine this, the application may use a rules engine and/or algorithms. In another embodiment, the application may apply a value to each piece of medical data and then determine a likelihood that the clinician would use this in an analysis of the data to determine patient health, changes, or important indicators of medical events, medical conditions, disease, abnormalities, etc. Data that meets a minimum or maximum threshold value then is ranked or prioritize as information likely to be considered by the clinician. The application may determine that applicable medical guideline(s) would make the information likely relevant and rank the information based on the guidelines. The application may then use the information and determine a graphical display that may be viewed by the clinician. Through the determination process, such as using the rules engine, the parameter may immediately become part of the display. The determined display may then become viewable at a graphical user interface of a computing device, providing the clinician with insights based on the existing medical guidelines. The application may display some information of higher value to a clinician and not display other health related information. For example, having the patient may have broken their toe as a child, and they information may not be highly likely to be related to a heart attack at the age of 75, and thus, the determined display would not contain the toe information.

In one embodiment, the system may have inference capability. The application determines risk of medical conditions or events and reports the information. In one limiting embodiment, EKG and blood pressure of 124 over 76 data may be part of the application determination. The application then reports back that the patient has pre-hypertension and is at risk for developing hypertension. The reporting may be done in real time with the patient present or without the patient present. The reporting back may be that the application takes the information and includes the information in the display. In other embodiments it may send a message to an account user, or it may send a message visible brought the application. The information may use a series of cardiovascular nomenclature to indicate the status of the patient from a cardiovascular disease management standpoint with a templated output which can appear as a pre-drafted medical note in the patient record. The information may allow the user to enter a note about the patient's health into the system that can then be used as part of the analysis. The reporting of the risk potential may be determined in a variety of ways.

The application can access records that are not only saved within the application, such as data related to the patient's health and the user account information, the data may also or alternatively be saved in a system that is accessed by the application for data retrieval. The application may access one or more other databases for information. In some examples, patient records databases may be accessed. Databases that include information about patient conditions, pharmaceutical drug databases may be accessed, medical records databases, rules engine databases for operation of data analysis, healthcare guidelines databases, and research databases, as well as other databases that may contain information about the care of patients.

What then happens to data is then organized in a visualization output of the dashboard. Processing of the visualized data and other data may then calculate a health score. A health score is a holistic number result after a holistic approach to taking all data and factors into consideration of the patient, and then output a number associated with the parameters of the individual patient. There are more than 50, such as heart conditions, habits of the patient, specific about physical body, body signs, vital signs, bio markers, results of recent diagnostic (EKG, imaging, blood tests, physicals, etc.) and other medical problems that people have that impact the management of heart disease. Genetics may also play a role in the health of the patient and play a role in the patient's health or disease, as well as the response to treatment. Graphically displaying severity of all the conditions in a signal plot may allow the clinician to interpret the information if the patient is dramatically ill or well, or to see if the visual progresses over time and to track on the graph a trend over time of the patient's health. Specific elements in visual identify if the patient is getting better or worse, and the visual may then be distilled down into one score that may track not only the progression of this set of characteristics that is used to describe the characteristics of the patient's health. Score is calculated based off all the critical parameters displayed on a radar plot. In some embodiments, many medical conditions contributing to the patient's overall health and the severity of the conditions.

In some embodiments, the application may take health related data and may modify the health score and medical related determined output. Different conditions may tend to have a variable degree of impact of health, life threatening, severe and can be managed, and other conditions can be managed. By viewing the health score, the user may know that these conditions will have an overall impact on overall health of the patient. Incorporating for relative contribution into the score. Beyond the score, additional data graphics maybe, in real-time, updated within the dashboard. In this way, an area of data may be updated or a portion of the area may be updated in real-time. Thus, using these indictors to help prioritize and learn important conditions and changing the health score may allow the application to determine an output likely to be used by the user.

Prioritization may use machine learning and AI techniques to allow the application to prioritize data and create the output for the user to view. The data may determine that certain pieces of data may apply a value to the data, based on clinician use, relevancy, guidelines, rules, etc. and may contribute to the determination of the overall value or group of data that has a value. The data is then used based on value and relevancy. The application then creates the output based on the values of the data, presenting the information likely to be the most relevant useful to the user. The user can request that the graphical output show more or less data. The user may also enter the relevant information for the patient into the dashboard or patient record, and may be included in the dashboard and the entry becomes part of the application's analysis.

Graphic may determine how a patient looks at any point in time, as well as the ability to compare that to previous to determine a relative variation of time a to time b. In one embodiment, the user of the application may determine globally if someone looks subjectively the same—stable, or if the health score looks about the same. A rules engine is—stable, and to look at various features that contribute to the features to see if they are improving or getting worse. Result in a balanced movement some getting better and some getting worse.

In some embodiments, the heart score or health score may be part of clinical decision making. The score may, in some instances, remain a static number. But the visual will show how the patient changed over time. When the number is worse than it will show you how it got worse. The number will show you how it got better then it will show you how it got better. It will look at the 50 some features contributing to that health score. To identify if some things are improving or getting worse. Results in a balanced movement of the features, some improving an some getting worse. The score may not change but will show better or worse in certain areas and will point out to the physician areas that need attention even though clinically they are “stable.” Score is a static, value calculated on any one point in time.

Graphical display of the data will look like a radar plot. The radar may, in some embodiments, include a health score, but the core may, in some instances, remain a static number. Although, the visual may show how the patient has changed over time, if it's gotten better then it will show how it's gotten better, and if it's gotten worse then how it's gotten worse. The visual will show this because the previous to present will show the change. The overlays will show the different point in data.

In a first embodiment, the application may look to medical records data. The application may apply values to the different types of data and information within the data, so prioritize data and identify the likelihood that the data is applicable in the display. For example, more recent information may have a higher likelihood of being used in the health chart than a cold from decades ago. Prediction techniques are used and values are compared against a threshold to determine if the information is likely to be useful. User input may also be used to determine the usefulness. The medial data may have a value or plotted indication of the value on a (radar) plot to represent the value. When more than one indication is noted on the graph, the application may determine shading in the area between the indicated values to help visually represent the area of health. The representative shading may show an indication of health is more or less concerning in a certain area. Colors and patterns may be applied to further highlight and distinguish certain areas. The areas indicated help to show quickly an area of health, relative values and test results, with respect to certain medical records, test results, measurement, etc.

Once the graphical image is created based on medical records, or past data, the application may look at current data, such as diagnostics, current inputs, device readings from a clinic or a home or remote application, notes, or other real-time and current data. The application may then create a graphical overlay, changing the graphical display, indicating the current readings as a visual to see how the health of the patient has changes from the past history. In one example, data points may be plotted, more than one, to represent the area of shading. These data nodes help to create a boundary of points. In some instances, the nodes may be at the same point as the past indicating that the health of that parameter is relatively unchanged, or in other instances, the node may be located at a different location, indicating a change in the parameter or health of the patient. Due to any changes in nodes of current parameter values, the resulting image may indicate the health of the patient has changed due to the shaded area having a different shape or form and different nodes being in a different location. A change in shaded area may indicate a change, for better or worse, in health and may have an association with the health status and overall health score of the patient.

The application then stores the determined output in association with the graphical output for that patient. This data may then be accessed for further visits and future use of the application.

It will also then become part of the “past” data as visits, diagnostics, device data transfer, and further medical health reporting. The data is recorded with a date and time stamp so that the time and medical changes may be tracked. Date and time data may also allow the user to view data of a particular date and/or see medical history on a date or within a range of dates. The data may be entered later than the actual date of record, but the system will organize and prioritize the information within the correct sequence and recalculate scoring accordingly. Similarly, if data is corrected, then the system will recalculate scoring to set the data in a more accurate determined result. The control of data allows the user to precisely view data to understand how medicine or therapies are working or other treatments. It also allows the user to see how disease and health is changing or effected by outside influences. The date allows a controlled way to look at specific information at a specific period of time or over a period of time. In other embodiments, the user may look at only the diagnostics and select specific layers of data to review. Selection may be the user choosing a date, type of diagnostic, parameter, date range, type of data, diagnostic, etc. and the graphical display may modify, or filter, the graphical display to output only the selected items and displaying them at the graphical user interface. The selection preferences may apply to the entire dashboard output, or the selection may apply to only a portion of the dashboard, such as the radar plot, and the other dashboard information may still include the none selected information.

The application uses prediction techniques end visual of the dashboard may represent cardiac medical records, diagnostics, medical records data, the radar plot of health. and other possible health associated information. The dashboard may provide a multidimensional longitudinal dynamic health state in a single graphical chart. The dashboard may also allow for patient similarity assessment if it is compared within a population set of similar patients for assessing any recommendations based on care provided to the other patients within the same population set. The dashboard may take large amounts of data, determines the most useful data, and outputs it in an organized graphical output that allows the user to see many different areas and medical considerations. It saves the physician time, uses important health data reducing the likelihood that something is missed, and gives the user an indication of health progression.

The dashboard goes beyond the typical medical practitioner's linear data plots over a period of time. This is different because of graphical output and understanding trajectories, because of all these variables, we aim to predict over time what the next visualization will look like and what the resultant contributors will look like. A layering may show a progression and prediction. The dashboard also gives an indication of the patient health score and health (score) change.

To use this information which is specific to the patient, the graphical display may be more accurate than displaying only the overall (and possibly generic) risk of heart disease, that could only be based on a general overall large population, without consideration of the patient in front of me). The application may provide a way to show or frame the health score that is more intuitive and logical. The dashboard presents all prioritized information about the patient's health and presents it in a graphical output that is organized and useful for immediate understanding of the patient's health.

The RPM Alerts dashboard is additional warnings that are tied to a specific patient's threshold(s) for which the physician needs to be alerted for when the vitals parameters are reaching or went beyond the thresholds set. This allows the Physician to prioritize a group of patients that are showing more extreme signals compared to the normal feedback shown by their health statistics.

The system allows for a more improved and ergonomic methodology for patient enrollment and device mapping. This approach allows for the enrollment and device mapping to occur in a single framework occurrence. The benefit of this system is that the approach tends to differentiate between devices and enrollment for multiple chronic conditions using the same device or other devices with similar parameter tracking. This ensures that the system will be flexible to the requirements of the patient or organization on how they want to enroll and track the health of the patient.

The system through its Natural Language Processing (“NLP”) and integration with EHR systems allows for the auto population of the Assessment and Planning (“A/P”) section. It will also indicate the elements of the A/P and how that impacts the risk score in a positive, neutral or negative manner. The system may help prioritize which of the planning elements will make the most impact in improving the patient's health. The text extraction and NLP allow the system to process the various data inputs and historical information of the patient and help formulate the needed assessment and associated plans\recommendations to reduce the risk to the patient's health in a prioritized manner.

The dashboard may also display graphics that show an intuitive and predictive health when the patient is treated or when parameters are changed. The radar plot, diagnostics, etc. may provide information to the user clinician for assessing how the patient would likely be impacted, based on patients that are similar to the patient associated with the dashboard display. Using prediction to determine patients similar to the identified patient, and then looking at how results of those patients when treatment changed or when parameters changed. Using data from similar patients, not the general population, then the application may more accurately predict that impact on the patient's health. The doctor or clinician may also be able to identify areas that may have an impact at certain stages of health or treatment. The application may then predict how modification of parameters, and certain order of modifications, may result in a more beneficial result for the patient. The collected data of similar patients may include information that the application may use when determining the likelihood of success when a parameter is changed, or in a sequence of modifications are made, and may enable the application to perform a statistical analysis of health of predicted result associated with the patient's own health and likely resulting health. The application may have higher thresholds for determining stricter requirement, to limit the number of “similar patients,” to improve the prediction of the patient's own health and response to varied treatment. Likewise, the application may reduce thresholds to gather a wider range of data or give a priority to certain parameters to determine “similarity” of health. Only through computer advancements and the increasing collection of patient records is this type of data and heath impact possible. The filtering of the data is also necessary, due to the large volumes.

In one embodiment, the information may relate to the population and may identify a variety of other patients similar to the patient that the user is reviewing the information for. Similarities may be based on medical records, physical condition data, diagnostics, genetics, and other characteristics and parameters that at least one other person has that is similar to the patient's associated data, medical history, health records, location, diagnostics, habits, treatment, account information and other data relating to the patient.

The application may assign a value to each element of the clinical medical data. Natural language techniques may be applied and values give to terms identified. Measurements, parameters, and results may also be assigned a value and weighted base it on the likelihood that a clinician would likely use it in determining patient care. Thresholds may be applied or value ranges to determine if the element of clinical medical data scored enough to be used in the determination. then determine the likelihood of those similar and look at their treatment and the outcome of changed parameters, treatment, time, changed behaviors, etc., the application may then determine a graphical output of the “similar” patients and graph the results. The determined output of the data associated with the other similar people/patients may then be used in the graphical output so the user can see the results and use this graphical display for analysis and comparison of the patient's data. The determined graphical output may have a series of dots or a shaded area representing the area of health results.

In some embodiments, the application may use the data associated with other patients to determine an outcome of the patients and the similar characteristics may determine a likelihood of health change similar to that of the other similar patients' resulting health. The application may then use the determined output to update the patient's dashboard with the results of the similar patient's output. In some embodiments, the graphical output may change to include a layer of similar patient data on the radar plot that overlays the patient's data on the radar plot, so that data layers are represented differently (colors, patters, etc.) and the user may then see where the difference of data is visible. In one embodiment the radar may have graphical areas identified and modified on the radar plot. In other embodiments, single points of data may be modified. In yet other embodiments, color modifications may occur to highlight the changed areas.

In other embodiments, there may be call outs or sub windows that zoom in on the changed areas of interest. A user may select an area specifically, to get more information on the identified area. In some embodiments, the user may click on the changed area, and may be able to zoom in or cause a sub-display to occur with more detailed information. In this way, the overall dashboard is kept simple, and the user can indicate that more detailed information about the particular area is desired. Hovering may cause an area to automatically enlarge or show more data related to the area near the hovering of the curser or user touch input. The user may click, hover, or select an icon that caused the display to include the more detailed information about the area selected by the user. The selection or input by the user for requesting detailed information may then update the application that the user is interested in the information with regard to the patient's records and treatment of the patient.

Modification of the graphical output with comparative similar patient info may help to provide graphical information about factors that also may impact care for the patient in front of us. The user can see treatment, lifestyle changes, factor influencers, impact over time, etc., and see what has positively and negatively impacted health. The user may then see what would likely impact the health or state of the patient's health represented in the dashboard. Drug trial data may also be used to indicate factors that may help determine what outcomes would be the most successful for patients and what would impact their health. Clinician look at research trials and may make choices based on trial outcomes, such as if 60% improve and 40% may get worse or not change. In this way, it may look as if the treatment was superior than not doing anything because more than 50% got better. In the example of looking at beta blockers in a large population, the beta blockers may work for one person, but data may not be available about people who are similar to the patient being treated both medically and physically. The application may consider how the other similar patients did when given beta blockers. Comparing the data of all those that took beta blockers and using those likely or most similar to the patient to show treatment results and trends may be used to determine a likelihood of success for the patient.

Many methods may be used to determine the similarity of other patients by comparing them to the patient's physical, medical, geographical, genetic, etc. status and then use these methods, such as predictive methods, to find these patients. A determined similar patient may have their medical data kept private in the applications, so that privacy issues are not created. Only the parameters, medical record, statistics, and other related predictive outcome may be used or indicated in the output of the application. The application may then be able to execute comparisons. In one embodiment, comparisons may include the data of the dashboard such as the radar plot. The application may include parameters and the variables, including values or representations of each variable. This variable analysis may then allow the application to determine identification of other people in our population that have values in our same range. Identification may include a 0-10 percent variation allowable for determining “similarity” of the other person.

In this embodiment of the dashboard, the radar plot may include updates or a separate radar plot may be part of the output. The user may indicate instruction that the graphical output may show the data associated with other patients and likelihood of outcomes with the patient associated with the dashboard display. The modified or selected radar plot may include the graphical output data that includes similar patient information and overlay and calculate areas of variations. The updated graphical output then may indicate an identified similar population health and results of changed factors or parameters associated with their health or condition. To illustrate the area of the radar plot graphic or the dashboard graphic may have an overlay area of a different color or pattern indicating the similar patient population parameters. The overlay may represent a list or multiple parameter values for other patients and may be represented in a graphical format overlaying the patient's historical data. The graphical area may in some embodiments represent more than one parameter and may be based on more than one other patient. The graphed area may also represent a likelihood of similar patients and a likelihood of patient outcome associate with a parameter or set of parameters. The graphed are is then placed in association with the patient's resulting output of medical record data to indicate real-time likelihood of medical attention with different parameters, which may indicate likelihood of success or reduction of success with each parameter.

In some embodiments, the system may identify new parameters or other parameters. The application may find the parameters in the system and in other databases, or entered by the user. The application may then use the additional parameter or parameters as part of the determination of patient health. The application in the system may update the rules and determination techniques with the additional parameter. User input may also allow the application to learn new data points to help the application learn and update and improve predictions.

The determined overlay of other patients' medical information in the radar plot, as well as other dashboard information may be updated or modified based on real-time data acquisition, note entry, procedures, and diagnostics being run on the patient. The updates of past record information as well as current and real-time information will update the graphical representation of the data shown on the dashboard. The application may create an output that organizes the data in a useful manner and show different aspects of the patient's current health and changes. The application shows all the heath data likely to be used by the user for patient care. The possible additional parameters or changed action data may change the determined output that is displayed on the graphical user interface of the computing device. The updated output, which may include the predicted modifications, may then include an output overlay with the predictive data organized with the other diagnostic information, which may be modified if associated data updates occur. Some additional system data that the application may receive and include in the output determination includes, but is not limited to, future trend of the treatment and likely responsive success of the patient should similar treatment be provided to the patient. The likely responsive success would be based on trending of other patients with similar parameters. The application may analyze effectiveness of surgical intervention, or effectiveness of medication, how the outcomes are impacted with multiple factors considered in the determination.

The records and medical history create a representation at a moment in time of the patient's health. Looking at other system data, however, makes the output of the data, that includes the health history layer on the radar plot, population outcomes, and then looking at the data study of similar patients (e.g., maybe 15 patients or another amount). Trials may focus on large populations that benefited, but there are those in the trial that didn't benefit, and so the user may use data related to knowing data from those that did not benefit as well. As a non-limiting example, if a beta blocker would likely be recommended for a heart attack or person with a set of medical conditions in addition to heart disease. Guidelines may instruct providing the beta blocker, but if the person has asthma, then they do worse on beta blocker, because the patient's breadth may be affected putting more stress on the heart, and paradoxically it may increase the patient's risk of having another heart attack rather than protect them from one. The radar plot may show all people with coped or asthma and I want to match against those and no one else. Radar plot has 5 rays with same conditions, heart attack asthma and kidney disease, etc. and may within range 5-10%, or they are 85 and above +/−5-10%. They are on metoprolol a mg dosage 5-10% and give you the overlap with 10 patient and show who is on beta blocker and who got worse from asthma. It shows what happened to those that looked similar to the patient. The user may see likelihood of result of giving a beta blocker to the patient and seeing possible counter indication because of other parameters, or conditions such as asthma.

In another embodiment of the invention, the application may receive other. Use databased of drug classes and interactions, and so seeing a likelihood of result associated with pharmaceutical intervention may also be possible. If a cardiac patient is on cancer medication, the prescribed post cancer medication may impact cardiac treatment. Thus, the system may include medical record databases, so this valuable medical information may be considered. Thus, the application looks at cardiac health, and the application may consider other information, too. In other embodiments, in the situation of cancer medical data that may impact patient health, other medical conditions may also impact how the patient may likely respond to medications or other treatment. Additionally, new medication, treatments, procedures, etc. become available over time. Medical related companies may have new data about treatment and new studies that may also provide value into likelihood of success for the patient associated with the application executing on the computing device. Thus, the system may update with new and updated information available to the system and possibly also validated as reliable information.

Thus, the application updates the information with modern and past medical information. Through access to the information, the application may show what is good not for a number of people in trial, but what is best for this patient associated with the dashboard information viewable on the computing device. The prioritization of the information helps to organize a display that likely shows info for the best decision at the right time for the patient. Access to the dashboard, showing the information at the point of care, may display information that illustrates health care that is likely to lead to favorable or positive medical condition direction. The application may use cardiology parameters in one embodiment, with parameters considered by practitioners in cardiology and cardiac health, and thus, the application may determine information based on cardiac clinicians and may determine and output of the determined information, pulling the information together, that is likely useful in cardiac health care treatment.

Natural Language Processing (“NLP”) may be utilized in one nonlimiting embodiment in conjunction with text extraction to pull critical parameters based on defined custom entities from unstructured or structured text. The unstructured or structured text may reside in documents such as general patient charts from Epic, FHIR data which is discretized, Lab reports, Echogram, Cath, Stress and ECG reports. It uses components of NLP through trained AI Neural Networks especially in looking at relationships of meaningful data and normalizes them into discretized information which gets plotted on the radar plot. The application may set operation in a way similar of how clinicians would use the data or would likely use the data. What did it tell us? What the clinician would find useful and act upon. We show the information and show similarities and the trends. Showing where we think things are headed and where they are going. It doesn't show treatment of the patient(s), but it shows cholesterol is 180 two months ago and its 220 today and it will be 250 in the next month or two. The system may show this and it may output statistics, but does not show treatment. The unstructured text may be classified in the Database or Health Data Lake as raw original documents as well as the codified information either within the specific terminology used by network server or the equivalent ICD (International Classification of Diseases), UMLS (Universal Medical Language System), Medical Terminology etc. The unique aspect of the system is that will keep the specific area of extraction and display both the raw original text as well as the extracted information.

The radar plot may change with the continuous updating of medical data input. Update prediction constantly with most recent data. User gestures at the graphical user interface may have effect of display and how to display different layers of the radar plot multi-layer display. The clinician may enter data into the application, such as updated scores, notes, request views of certain medical data, etc.

The Health Score will change based on new data points that are affecting the risk calculator and formulae including weights applied for the different risk factors—See Table 1. The Health Score is a formula that is developed based on the overall effect of the risk factor to the person's health state and takes the shape of a particular pattern.

Baseline Risk from Validated Models (such as Framingham) Score+(Risk Factor 1*Weighted value+Activity factors)+Medical Conditions and characteristics+Social Determinants of Health (Socio Economic Status)+Activity+Financial performance=Normalized (Lower Number (Poor Health) or (Higher Number (Better Health))). The color of the Heart Score ring also indicates the health status of the individual that is represented by the Heart Score. The Heart score may be dynamic in nature and changes based on data inputs from historical or new tests. Every data point based on the timeline of the update and whether it has been applied to this score may reflect a change in the Heart Score.

The example used to describe the invention herein is cardiology. However, the invention can be used to graph data in other areas of medicine and wherever data graphical display outputs are used to illustrate a wide range of data in one organized view, showing progressive changes. It may be used in other areas of medicine to see the patient's health change and to understand how other patients, with similar characteristics, have done over time as well. Areas on the dashboard may indicate that the user can click, highlight, hover, etc. the area and view more detailed information regarding a certain period. In other embodiments, the overlap view of the radar plot may change when the user indicates or interacts with the user interface to remove or add the layer that may show likely similar patients to help identify the likelihood of improvement or worsening of the patient's condition when modifications occur. Modification may include, but are not limited to changes of: treatments, parameter, life style, other disease, health, diet, stress, genetics, medication, etc.

The application is an organized graphical display that allows the user to see the records and reads of the patient as well as the likelihood of improvement or decline when parameters change to help determine a risk determination. Determinations and calculation of prediction likelihood of similarity to patient; prediction of outcome of patient based on other similar patients with various treatments of factors.

The rules engine may allow the user to add all the risk factors and associated information for processing certain inferences to allow the physician or nurse to have additional insights to the status of health and how they could improve the health based on current guidelines provided by the MIPS (Merit-Based Incentive Payment System). The MIPS guidelines provide clinicians and health organizations standards for treatment to follow based on factors such as age, medical conditions, risk factors seen.

The mapping engine design may allow for the user to establish routes for the data ingestion followed by processing guidelines which then renders into a specific portion of the user interface or other parts of the system platform as needed. The codeless initiative allows for the data to be agnostic in nature and can come in multi-formats and data access standpoints. This includes, JSON, XML, FHIR, HL7, DICOM, PDFs, JPEG etc. It also allows for the data to be transacted in variety of manners such as HTTP, access points, TCP/IP and special ports, email exchanges, data uploads manually. Overall, the mapping engine allows you to set the input, the processing mechanism and the output without having to hardcode this information into the platform.

The database design may be developed with the intent of storing data in a time relevant manner and also support a high-performance design for addressing improved querying of information with a SQL (Structure Query Language) like query language. The database may also support the ability to store information in a JSON (Java Script Object Notification) like data structures which are easy to chart natively.

The precision health elements of the system design will be additionally incorporated to the main system architecture for the purposes of research and assessment. It will also be tied to the Machine Learning and Deep Learning operations by providing research teams with the option to anonymously review data to see patterns related to specific diagnostic trends. This will also help feed into the learning and implementations of this learning back to the patient similarity and improved recommendations aspect. The design components of the precision health system will include the use of native graph databases which are fundamentally more accurate in showing relationships with data in a performance-oriented manner. Additional elements of data may be used to improve the deep learning by including genetic genotype and phenotype data to improve recommendations and prioritizations. The application may also allow for pharmacogenetic information to ensure that the appropriate medications are recommended based on the current advised list and how the interactions maybe hurting or helping the patient's health.

The disclosure uses many non-limiting embodiments and other possible embodiments are considered. The disclosure describes the embodiments with a cardiology context, but the application may be used with many different types of medicine, not just cardiology. The disclosure herein uses the singular form, which also contemplates the plural, and the plural form also considers the singular form.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention.

### DETAILED DESCRIPTION

Generally, the present invention relates to a graphical display of medical data that allows the clinician to read all relevant data regarding the patient's health and update the data in real-time with predictive outcomes associated with the patient's current health. The graphical display displays an entire medical record of data in one dashboard with a radar output of health entries that correlate to the medical records of the individual, and output a health score associated with the overall heath entries. The application is an organized graphical display that allows the user to see the records and reads of the patient as well as the likelihood of improvement or decline when parameters change to help determine a risk determination. Determinations and calculation of prediction may indicate a likelihood or risk of medical conditions and risk, or a likelihood of a clinician determining certain health care plans to treat a condition of a patient, or in some instances, to someone similar to the patient. In some instances, the system may use prediction of outcome of patient care, based on other similar patients with various treatments of similar factors. Turning now to the Figures which exemplify parts of the provided system and methods,

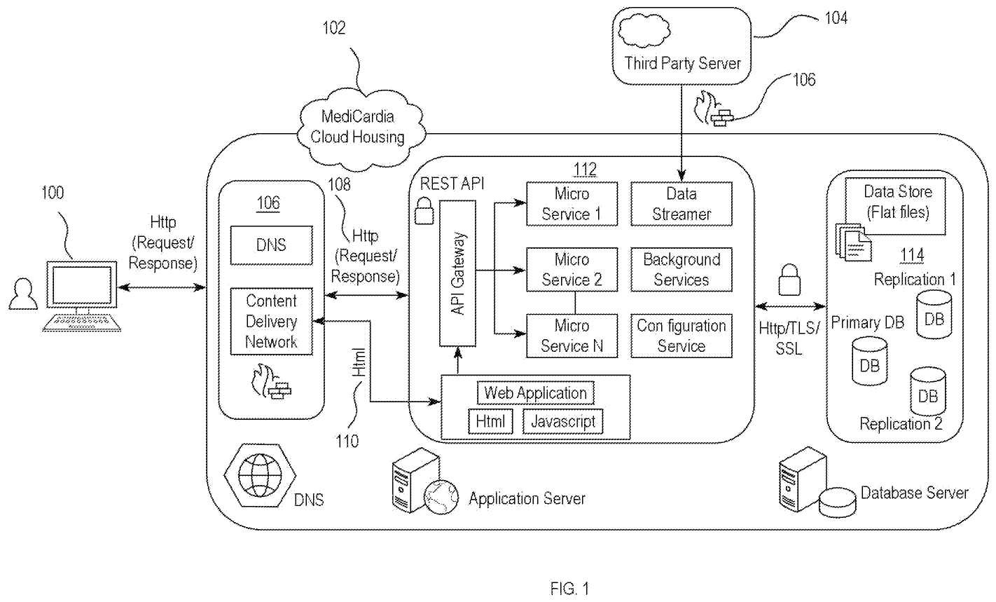

FIG. 1 is flow chart of one embodiment according to the provided disclosure where the system creates a prediction of health care and health data predictions tailored to the patient. FIG. 1 is an example illustration of an application that may include a radar plot prediction that may be an application executed on (or in a system with) a computing device. The application may display patient medical information, comprising a computing device that has at least one processor, wherein the processors execute steps for medical data display data that a user will likely use associated with a preferred patient outcome. The application may receive medical data based on a plurality of health factors associated with a patient, and utilize computer components of the device or in the system. The system may use or communicate with hosted AWS cloud, data storage, API gateways, etc. for system integration and function. The application may further determine a health score based on the plurality of health factors and then determine an output based on the health score and the plurality of health factors. In some embodiments, the application may receive medical data based on at least one other patient with health characteristics likely to match the patient's health factors and then determine a second output, which may be a revised first output, based on the received medical data. The second output may include an additional layer or indication visual to the user at a graphical user interface that indicate a predicted outcome of health and predictive health score for the patient.

FIG. 1 is an embodiment of the system and workflow diagram. The clinician and user may access the system through a computing device 100. In some embodiments, the requests and responses to the system may be mad to the cloud housing, which contains a number of different points of communication. For example, the DNS and content delivery network 106 may send a response or request to (http) Web Application/html/javascript which is then sent to the API Gateway off the Rest API 112 that may be locked or have restricted access. In some embodiments, authentication may be required to access the data. Depending on the account access authorization, different clinicians may have access to different records and sources. The API Gate may communicate with micro service providers and communicate with may include data streamer, background services or configuration services. A data streamer may receive third party data from a third-party server 104. Due to the third-party access, there may be a firewall 106 present for protection. The access may be through an http (request/response) 108 (or in some embodiments Html 110) or data may from DNS and Content Delivery Network 106 to Rest API 112 and the reverse. be sent into the system. The Rest API 112 (interface and services) may communicate vie http/TLS/SSL with authentication with a file storage 114. That may include, but is not limited to, data store (flat files), Primary DB, Replication 2, Replication 1. Data may be requested and sent back to the API receivers in the platform for further determination and carrying out executed instruction or completion of services. The application may receive medical data based on a plurality of health factors associated with a patient, and utilize computer components of the device or in the system. The system may use or communicate with hosted AWS cloud, data storage, API gateways, etc. for system integration and function.

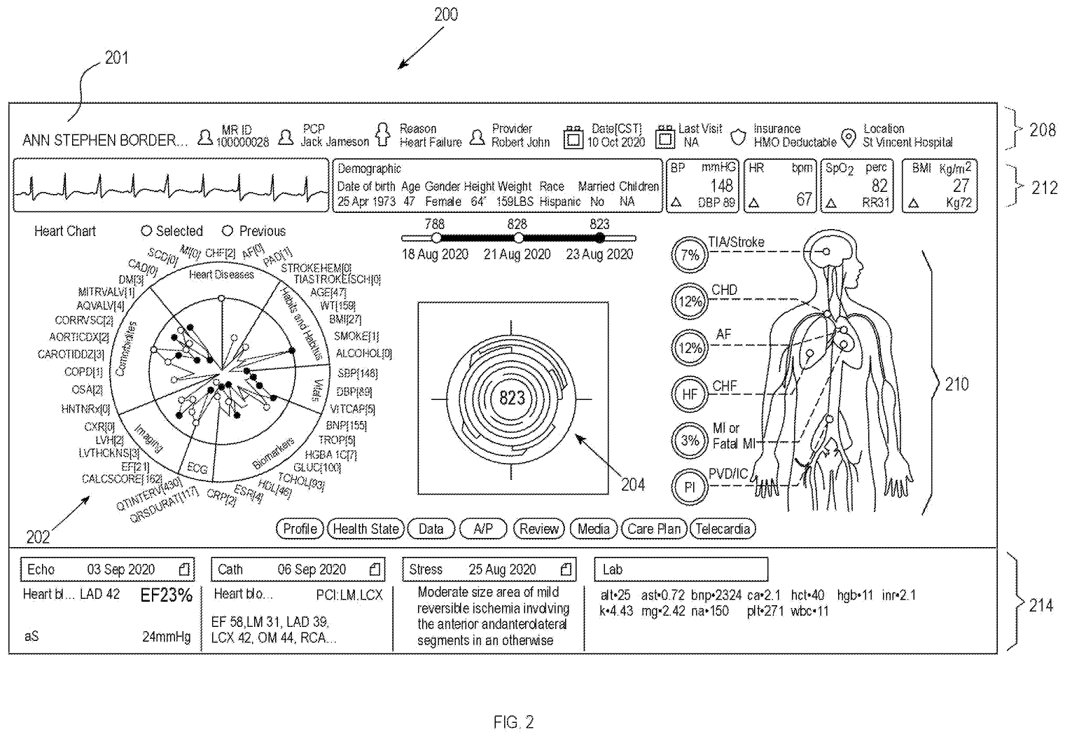

FIG. 2 is an embodiment of the dashboard that has medical record information 208, associated with a patient 201. Dashboard 200 may also have vital signs and demographic information 212 of patient 201. In this way, the information may be logically grouped together for easier and more logical analysis. The clinician, does not have to search for the information, medical record(s), stored or recorded, or test results, vital signs, and other medical record information may be part of a first output 200 (or in other words dashboard 200 or a portion of dashboard 200). Dashboard 200 may further include additional graphical visualizations of clinical medical data. For example, of an application that may include a radar plot prediction that may be an application executed on (or in a system with) a computing device. The application may display patient medical information, comprising a computing device that has at least one processor, wherein the processors execute steps for medical data display data that a user will likely use associated with a preferred patient outcome. The application may receive medical data based on a plurality of health factors associated with a patient, and utilize computer components of the device or in the system. The application may further determine a health score based on the plurality of health factors and then determine an output based on the health score and the plurality of health factors. In some embodiments, the application may receive medical data based on at least one other patient with health characteristics likely to match the patient's health factors and then determine a second output, which may be a revised first output, based on the received medical data. The second output may include an additional layer or indication visual to the user at a graphical user interface that indicate a predicted outcome of health and predictive health score for patient 201.

FIG. 1 may further illustrate an embodiment of the Medicardia Dashboard. In the illustration of the dashboard, the computing device may execute one point of application execution. The dashboard of FIG. 1 may show a radar plot 202 with multiple different parameters. The area in the middle may be a graphic representation of the patient's health. The indicated parameter or multiple parameters may be based on medical records and diagnostics, but are not limited to these. Dashboard 200 is a single panel look at multiple related determined output areas. Radar Plot 202, which includes multiple points of determined patient data, and analyzes multiple data points to determine a representative output. Radar Plot 202 risk variables of Heart Diseases, Habits, Vitals, Biomarkers, ECG, Imaging, and Comorbidities are noted in the pie shaped wedges of the circular graphic or radar plot 202. When the clinician clicks on one of these variables, the user is redirected to the ‘Health State’ window of dashboard 200, where the clinician can evaluate and predict patient ‘Cardiovascular Conditions.’ In some examples of radar chart 202, the data is normalized to appear related within scale to the other data points of the respective category. In this way, the data becomes more accurate and does not skew the visualization of the resulting radar chart 202, but logically contains the information for the clinician to use.

In another embodiment, a Heart Score 204 graphs the data and determines a value for the heart score representing the health of the patient. The changing heart score 204 may vary based on the year and that change may also be part of dashboard 200. Once in the Health State window, the clinician can view Heart Score 204 in the center of the window. Heart Score 204 provides an indication of the overall cardiovascular health of an individual. In general, Heart Scores range from 450 to 950, with lower scores generally indicating greater number or severity of disease or risk factors, or cardiovascular abnormalities, and higher scores indicating fewer number or severity of disease or risk factors, or cardiovascular abnormalities.

A Risk Score(s) 210 In this embodiment, patient 201, and a 7% change of having a stroke based on the past medical history and the acquired real-time data after determination, and determination and determined a 7% risk of TIA/stroke. that may also be determined by the application or by the system, as visualized with the anatomical indicators for each of the respective areas of risk. 210. The clinician system dashboard 200 automatically determines the Framingham risk prediction calculations in any, in this embodiment, cardiovascular “CV” category. This is the second output that includes these real-time predictions (based on current and past data) by clicking the patient's percentage values in the ‘CV Condition’ graphic. A pop-up window appears with additional details on the CV factor selected. In the pop-up window, the clinician can configure different risk factor scenarios—for example, by increasing or decreasing cholesterol or blood pressure values on a slider bar—to help predict the probability that the patient will experience a particular health outcome. The user may further interact with the graphical user interface of dashboard 200 by clicking the ‘Calculate’ button executes the risk calculation, the ‘Reset’ button (next to ‘Calculate’) allows the Clinician to reset the risk factor values to default, and clicking the “x” icon in the upper right of the pop-up closes the window. Test results and results 214 may also be seen as on the bottom of FIG. 2.

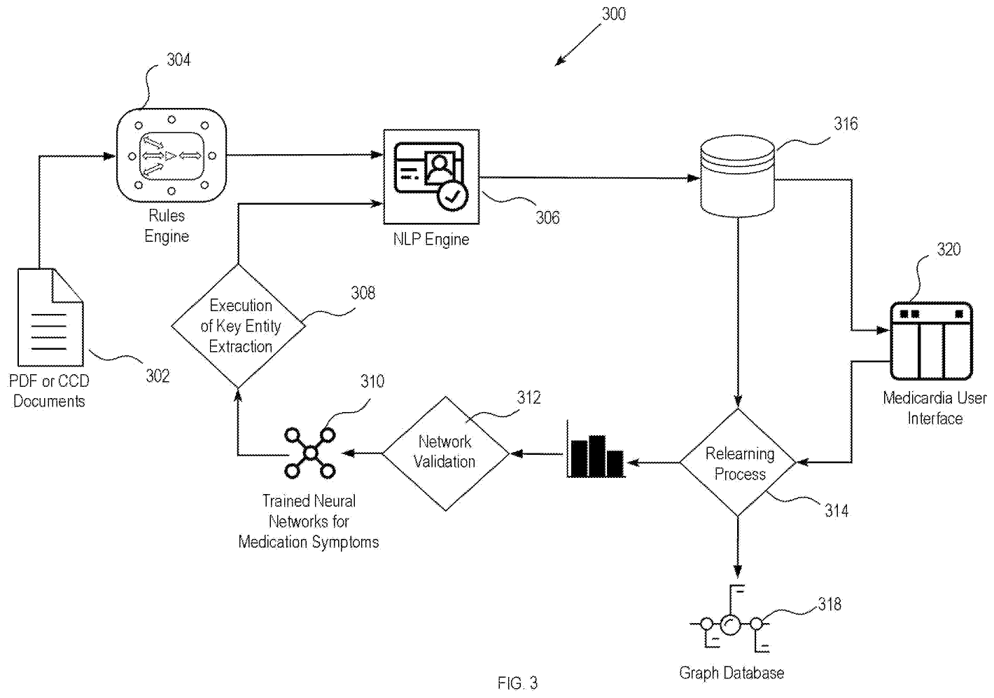

In FIG. 3, Provide a seamless NLP Engine with automated built-in retraining learning. In the embodiment of FIG. 3, a document or other data information may be sent to the system. A rules engine 304 will look at the text of document 302 and will put it theory an NLP Engine for analysis and determination 306. In this embodiment, rules engine 304 may apply information from a database (e.g. database 316) and application of rules for analysis and extraction of information and elements. In this way, the values or weighted information is already assigned may be applied to the element meeting the structure format in accordance with the respective rule. Information may then be received by NLP engine 306 for unstructured data processing were new or unstructured data. In this way, weighted values and data analysis of the elements contained in the data, such a natural langue, values, stored information, identification of data, test results, etc. may be analyzed and the respective element of the data received is given an associated respective score of value for the element of data. The data may then be stored 316 and output at the graphical user interface 320. For both structured and unstructured data, normalization techniques may be applied in the rules engine 304 and the NLP engine, or other areas of the clinical intelligence engine. For example, systolic pressure may be measured and the value may not apply to the graphical area such as a radar plot. Thus, system 300 may normalize the data and convert it to a scaled but useable amount to be used with the first or second output, useable in the determination of the respective output, and may also be used in the graphical user interface visualized output. In this way, system 300 may use discretization techniques to normalize and analyze the data for prediction and determination of the first and second outputs. In other embodiments, the system will store the information and put it bac into the system in a relearning process 314 and will add it to a graph database 318. The graphed results will then be put through a network validation 312. The information may then go into network validation 312 to verify and validate the information which is then applied into the trained neural networks analysis 310, which runs subroutines to determine medication and symptoms related to an illness. The information analyzed by the neural networks 310 may then go to an execution of key entity extraction 308. The extracted elements are then recirculated through the NLP engine 306 and sent to the user interface 320. This system may include an AI Training Neural Network model with base trained functionality. Allow for the user through the manual intervention to allow the feedback to retrain the model dynamically within the customer site. Allow for customization from site to site. Allow for version controlling of the neural network models to allow hosted networks to be re-versioned without any manual intervention.

Databases in the system, such as database 316, may be a storage device and may include a volatile or non-volatile computer readable storage medium that is able to store such as software programs and data to implement the functionality of the lane determination system. In some examples, storage device(s) may include non-volatile storage elements, such as magnetic hard discs, optical discs, floppy discs, flash memories, or forms of electrically programmable memories (EPROM) or electrically erasable and programmable (EEPROM) memories. For example, storage device(s) may include Random Access Memory (RAM), Read Only Memory (ROM), flash memory or any other form of long term or short-term memory, although without limitation thereto. In some embodiments, the memory may also include hard disk drive, floppy disk drive, tape drive, secure digital (SD) card, digital versatile disc random access memories (DVD-RAM), or any other appropriate form of computer readable storage medium [symbol] Processor(s) within computing devices is operably connected to a communication unit(s), an input device(s), an output device(s), a user interface (UI) device that includes a presence-sensitive screen, storage device(s) and communications channel(s). Processor(s) may also be connected to other modules/devices (not shown) within the computing device or connected externally via an appropriate interface. Processor(s) may include, but not limited to, microprocessor unit, graphical processor unit, digital signal processor or any other appropriate processors that have the capability to execute computer program instructions on data to produce the expected output. A processor(s), not shown in FIG. 3, may include a plurality of components from a list including registers, buffers, control logic, data lines, arithmetic logic unit (ALU), floating-point unit (FPU), and other appropriate components for performing operations including arithmetic, logical, control, input, and output specified by the instructions in a computer program.

Computing device(s) may also include hardware and/or software modules including antenna to communicate wirelessly to the Internet, a camera device to capture photo and video, capture audio, a call module, short message service (SMS) module, a media player module to play multimedia content (for example: music and movie), and an Internet web browser (for example: Firefox and Google Chrome), and medical data module for viewing medical files and adding echo scan data. Computing device may also have additional applications installed such as VPN network access, medical records, echo scan imaging and analysis, calculator, calendar, text editor, and other appropriate application programs.

In some examples, storage device(s) may include a graphics module, which may execute machine instructions or computer instruction to produce an output-on-output device(s) or send data to a peripheral device interface or other appropriate interfaces, and may use one or more of processor(s), which may be one or more from a list including single processor, multi processors, single-core, and multi-core processors. Graphics module may cause processor(s) to execute machine instructions or computer instructions to produce an output-on-output device(s) or send data to a peripheral device interface or other appropriate interfaces. In alternative forms of a user device, a plurality of hardware processors, types of memory, and data busses (not shown) may be present. Dashboard module may include prediction module and output module. Graphics module may send determined output graphical display data to UI module for displaying the determined content at presence-sensitive screen. In some examples, presence-sensitive screen is output device. In another embodiment, user input (UI) module may receive user input through one or more of input device(s), such as touch screen, audio, visual, keyboard, and other haptic based devices. The diagnostic module may execute instructions that include program instructions stored in memory within the storage device(s) (e.g., records, output, and data), stored externally, or transmitted by means of radio waves or electromagnetic waves. Dashboard module may retrieve device data from storage device(s), which is a data store for the computing device.

Patient records may be privacy protected, in some examples, enabling password protection for accessing the dashboard application or patient files. When a user enables scan module 58 on computing device, diagnostic module may send a signal out either wirelessly or through hardware to communication unit. Communication unit may send and receive data from communication unit(s) via communications channel(s) 50, sending and receiving data to the intended application or device associated with diagnostics. Communication unit(s) may receive data about the image or other condition identification related information (e.g., geographical data, family medical data, genetic data, population data, etc.) from a remote server via the wireless network or other appropriate communication network, such as communicating through one or more communication technologies including cdma2000, WCDMA, WiMAX, Wi-Fi, 25 Wi-Fi Direct, BLUETOOTH, GPRS, 3G, 4G, LTE, satellite based communication, and other appropriate communication technologies that will be known to an ordinary person skilled in the relevant art. (e.g., Wi-Fi, a peer-to-peer connection such as BLUETOOTH or Wi-Fi Direct, or other appropriate form of connection).

Further, Dashboard module may automatically determine a layout for the dashboard graphic. Dashboard module may cause processors to connect to appropriate storage device through dashboard module to retrieve and store data. In other examples, computing device may be connected to external devices through wired or wireless connection as appropriate, or through Universal Serial Bus (USB) or other wired connections. For example, dashboard module may include mechanisms to receive data or send data to a remote server, such as a medical records or medical data, medical guidelines, or medical information using known techniques.

Prediction module 66 may receive the image data and any other associated data associated with the user e.g., family medical history, geography, population data, etc.) during a diagnostic or test and prediction module may determine any indicators in the received data. Prediction data may associate with the graphical image of the ECG, echo, and other associated data may associate with the non-graphical data. In some examples, prediction module may determine a weighted probability for each of the determined indicators of the received data, for the likelihood that the received data is an indication of a condition, for example, prediction module may determine whether a probability exists for each determined indicator. If a probability does not exist between a suggested sharing service and the indicator, prediction module may generate a corresponding matching score, or probability. In some examples, a probability may be a value in a range between 0-1. In some examples, a probability may be initialized to a value of 0.5. Once dashboard module locates user preferences, prediction module may compare the determined data with the characteristics of the condition's indicators. Based on the comparisons, indicator module may determine the probability based on a comparison of probabilities determined for previously identified indicators, stored in a data storage. The indicators and the associated weighted probabilities may then be sent to output module. In some examples, only certain values or scores may be sent to output module. For example, applying a threshold value to determine which indicators to select and send to condition module for further condition analysis. A ranking or prioritization of the data sent may determine the graphical output.

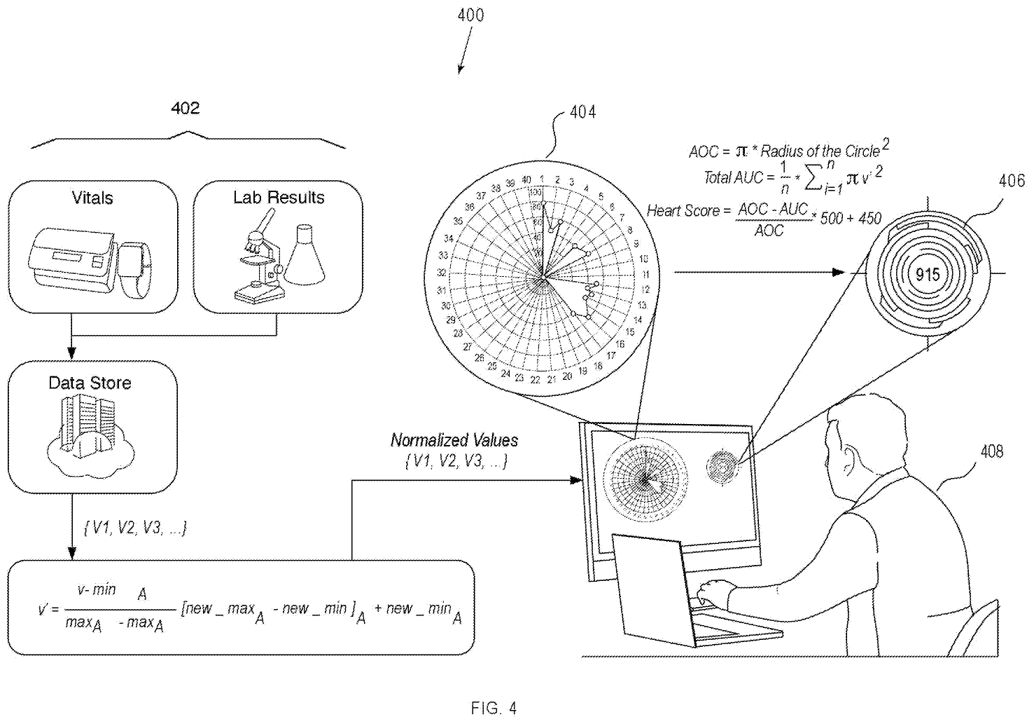

FIG. 4 is an example block diagram illustrating possible medical data 402 and determining of a radar plot 404 within the dashboard. The illustration is not limited to what is in FIG. 4, and may use other calculations and data points to determine priority and determined output for display. FIG. 4 also gives an indication of how medical data can be collected and put into the system, in a single usable visual format. FIG. 4 shows wearables, where the “wearables” data may be from over 300+ wearable devices are available for use in the system platform for medical data collection.

In the illustration of FIG. 4, which is an embodiment of a Radar Plot 404, that may show the risk variables of Heart Diseases, Habits, Vitals, Biomarkers, ECG, Imaging, and Comorbidities are noted in the six pie shaped wedges of the circular graphic. Further, in FIG. 4 may show a Heart Score. The Heart Score 406 provides an indication of the overall cardiovascular health of an individual. In general, Heart Scores 406 range from 450 to 950, with lower scores generally indicating greater number or severity of disease or risk factors, or cardiovascular abnormalities, and higher scores indicating fewer number or severity of disease or risk factors, or cardiovascular abnormalities. The information is in an intuitive format at a second output for clinician 408 to view in a logical and easy to understand format.

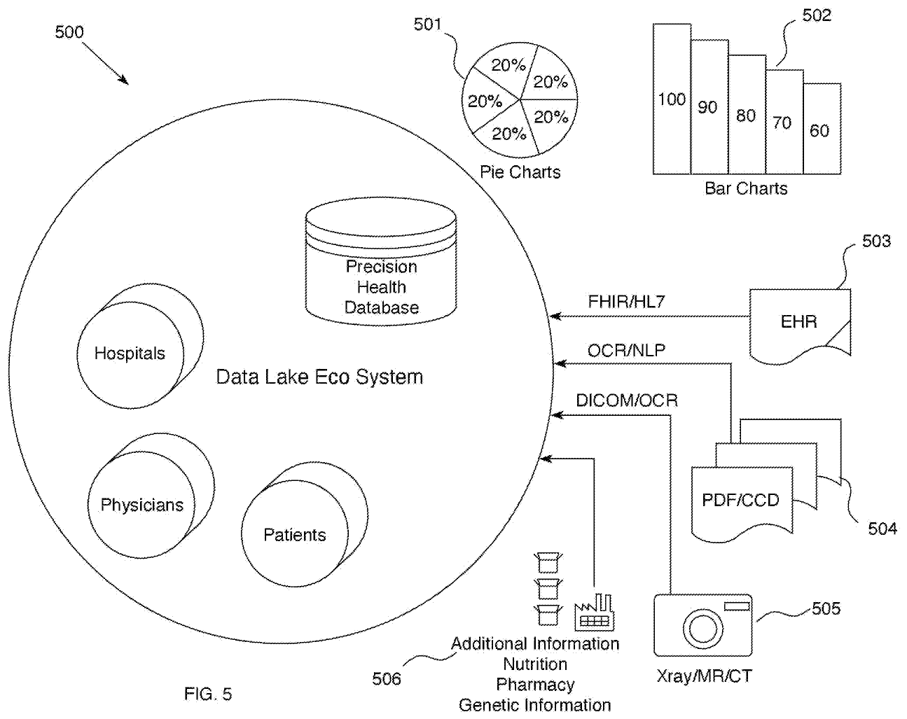

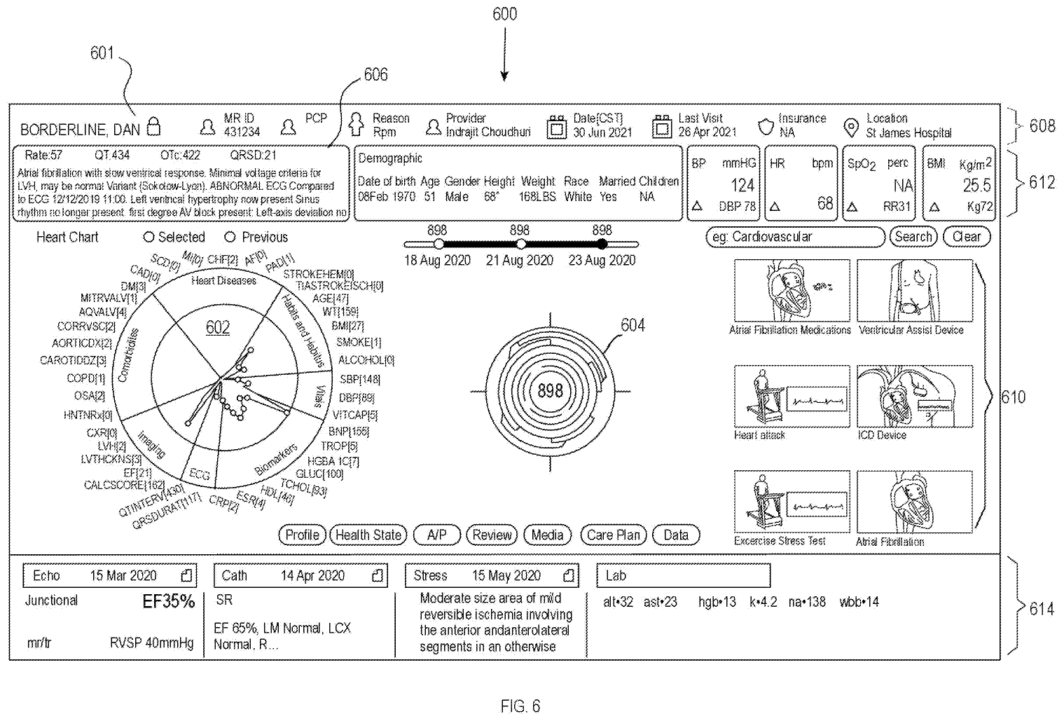

FIG. 5 is an embodiment of a data lake eco system platform 500 where hospitals data, physician data, and patient data, and precision health care database are all accessible. HER 503 may send data via FHIR.HL7 for determination of elements and where they should be sent and associated with. PDF/CCD 504 may be sent via OCR/NLP may also be sent Additional information such as pharmacy 506 or other information 506 may also be sent into the eco system 500. In some embodiments, pie chart 501 and bar graph chart 502 are examples of how the data may additionally, or alternatively, be laid out for visualization and understanding of large amounts of patient related data. An advantage may include a communication platform to combine video, text, email into a single communication overview. They may further allow for appropriate engagement approach that best suits the needs of the physician and patient, and the system may establish a coaching mechanism that supports the patient's ability to improve their cardiac health. FIG. 6 is an embodiment similar to FIG. 2 that shows a Heart Score 604 graphs the data and determines a value for the heart score representing the health of the patient. The changing heart score 604 may vary based on the year and that change may also be part of dashboard 600. Once in the Health State window, the clinician can view Heart Score 604 in the center of the window. Heart Score 604 provides an indication of the overall cardiovascular health of an individual. In general, Heart Scores range from 450 to 950, with lower scores generally indicating greater number or severity of disease or risk factors, or cardiovascular abnormalities, and higher scores indicating fewer number or severity of disease or risk factors, or cardiovascular abnormalities. A radar chart 602 graphs data according to all of the categories. The information has a wearable or implantable devices may also provide information, such as the areas of device data 606. Provide a mobile patient chart for the patient to help get a visual on the patient's health. FIG. 6 also shows test results 610 for medications, data from a ventricular assist device, heart attack, ECG, ICD device data, stress test, atrial fibrillation. Vital signs and diagnostics 612 may also be part of dashboard 600. Patient/medical record data 608 is listed at the top associated with patient 601. Lab results, test results, patient monitoring, procedure data, etc may also be in a grouping for easy to associate and read groupings 614.

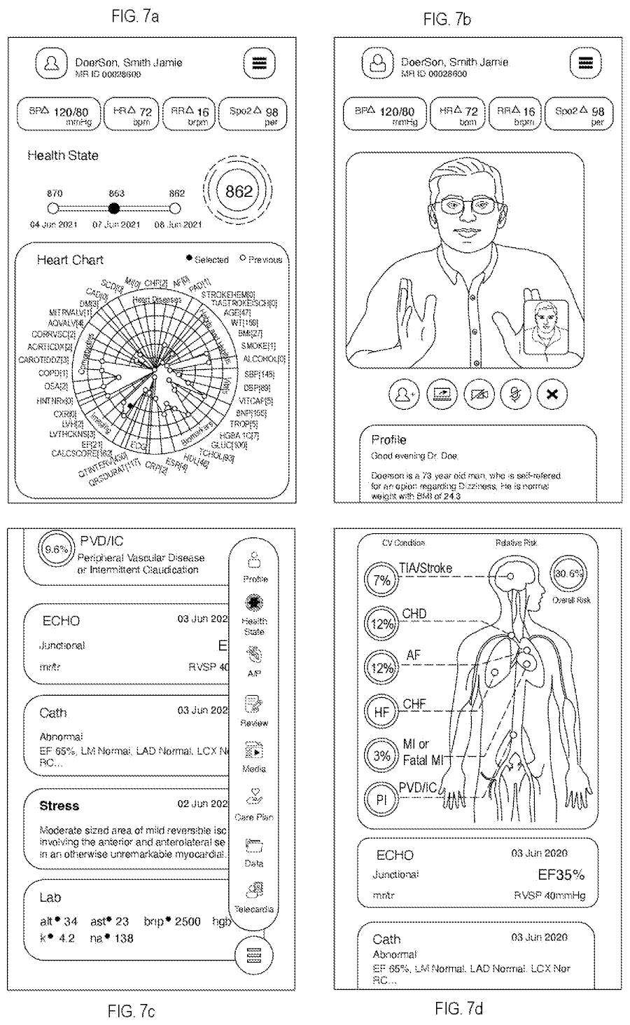

FIG. 7a is a mobile patient chart for the patent to help get a visual on their health. It is tailored to that information the patient as a user would likely find helpful. The heart chart shows the radar plot, health score, heath state indicator, and diagnostics (such as blood pressure, heart rate, respiration rate, and Spo2). The view of FIGS. 7a-7d are of a handheld computing device, such as a smart phone, tablet, or other display. There may be a risk score, that illustrates or identifies specific areas and what the risk of that medical or medical event likely may be, based on heath factors and diagnostics. Menu Options may also appear for the user to select what they would like to view or interact with. They can get more information about a diagnostic or a test. The user can update their settings. In another embodiment, FIG. 7b the application may have on demand video and texting or means to communicate with other physicians, clinicians, or with the patient. The view may include measurements and notes during the video conferencing, so that that specific medical information may be discussed. In other embodiments, FIG. 7c illustrates the videos may be pre-recorded regarding health information or the patient's profile may be viewable, along with medical history data to help with the discussion. FIG. 7d show the risk assessment based on patient medical clinical data.

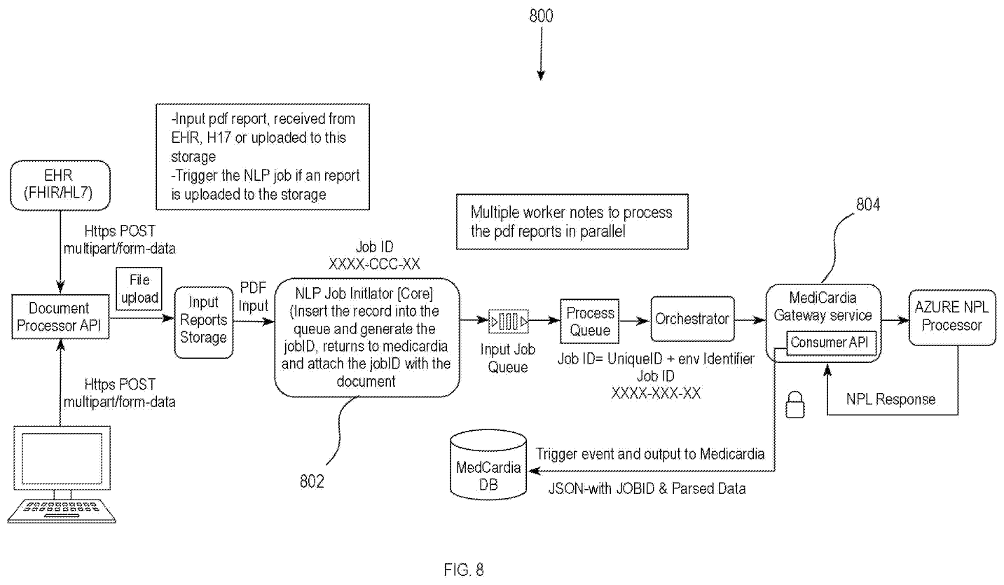

FIG. 8 is an illustrative flow diagraph 800 that shows an advance NLP diagram. It shows the input flow of information into the NLP Job Initiator 802 and into the gateway series 804 and output and storage.

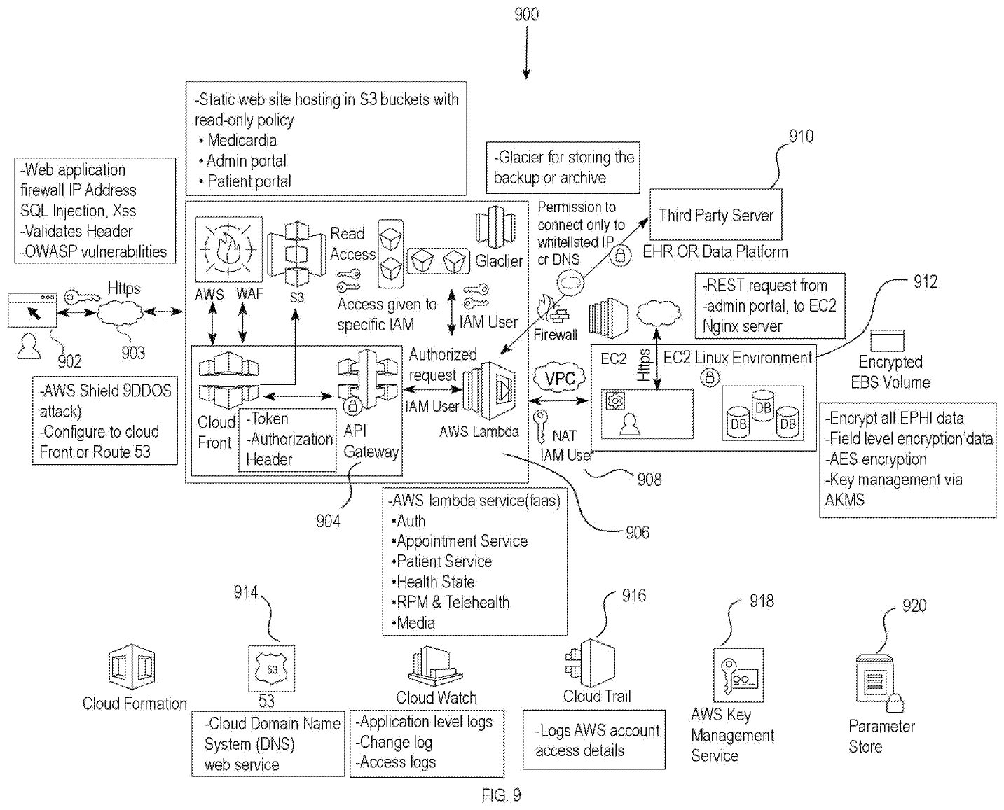

FIG. 9 is a high-level architecture flow diagram system uses the AWS platform to deliver a scalable cloud computing platform for high availability and dependability, providing the tools which enables it to run a wide range of applications. These tools and service protect the confidentiality, integrity, and availability of systems and data.

The system uses AWS 906 server-less infrastructure services to host the application and to execute the API 904 to get the PHI data from the server. Server-less infrastructure helps to build and run applications and services without having to manage infrastructure scaling and all the server management is done by AWS 906 with, in one non-limiting instance, Lambda protocols.

Security may be required with medical data and to limit false data impacting prediction, and thus, identity management access controls. AWS Identity and Access Management (IAM) enable to manage access to AWS services and resources securely. Using IAM, the system can create and manage AWS users 902 and groups, and user permissions to allow and deny their access to AWS resources. AWS account associated, in one embodiment, with user or clinician 902, represents the business relationship between the system and AWS 906, which is used to manage AWS resources and services. This account will function as the main organization account and has the root permissions to all AWS resources and services, the system organization might choose to use several AWS subaccounts, one for each major department/hospital, and then create IAM users, under the AWS account and then assign those permissions directly, or assign them to groups.

The system has different storage capabilities at different level of use. The application uses Amazon S3 bucket to store and retrieve any amount of data, at any time, from anywhere on the web as well as to host the system's static website which includes webpages, static content and client-side scripts. Amazon S3 provides highly scalable, reliable, fast, inexpensive data storage infrastructure that Amazon uses to run its own global network of web sites.

The platform provides flexible security features to block unauthorized users from accessing the data. VPC endpoints can be used to connect to S3 resources from a virtual private cloud. S3 supports both server-side encryption (with three key management options, such as AWS Key Management Service 918) and client-side encryption for data uploads. Block Public Access may include a set of security controls that ensures buckets and objects do not have public access. The system should not use PHI in bucket names, object names, or metadata because this data is not encrypted using S3 server-side encryption and is not generally encrypted in client-side encryption architectures.

The system application may use S3 storage for static hosting of web, to store patient reports and cloud storage is available, as is local storage.

Function may in some embodiments be a service. AWS Lambda 906 is used in the system to run code without provisioning or managing servers on their own. AWS Lambda 906 uses a compute fleet of elastic compute cloud instances across multiple availability zones in a region, which provides the high availability, security, performance, and scalability of the AWS infrastructure.

There may be a static hosting of websites include application by the system and admirative and patient portal. In other embodiments, there may be write access granted to an IAM User so a clinician 902 can write to a patient file and in a file log. Keys maybe used or functions may be triggered by executed action. In some instances, when other applications are used on the computing device, the executed application may trigger system actions.

Cloud services may be used in the system to enable a common platform to model and provision AWS and third-party application resources in the cloud environment. AWS CloudFormation allows to model the entire infrastructure and application resources with either a text file or programming languages, in an automated and secure manner, all the resources needed for your applications across all regions and accounts. This provides a single source of truth for all your resources and helps you to standardize infrastructure components used across your organization, enabling configuration compliance and faster troubleshooting.

The system 900 may use Route 53 as a scalable cloud Domain Name System (DNS). It effectively connects the user requests to infrastructure running in AWS—such as Amazon EC2 instances, API Gateway 904 or buckets. Route 53 914 traffic flow may make it easy for the user to manage traffic globally through a variety of routing types.

In some embodiments, the system may collect and monitor the operational data in the form of logs, metrics, and events, and visualize it using automated dashboards to get a unified view of the system AWS resources, applications, and services. Set alarms to monitor any unusual activities happen. Resources may include enable or setting an alarm for the activities, such as login, access, failures, error, and many more.

AWS Systems Manager Parameter Store 920 may be used in the system application to provide a secure, hierarchical storage for configuration data management and secrets management. Parameter store can be used to store the data such as passwords, database strings, Amazon Machine Image (AMI) IDs, and license codes as parameter values. The parameter store data has to be kept in a secured way and access only by specific IAM role user to encrypt and read from it. Parameter Store 920 may use AWS KMS to encrypt and decrypt the parameter values of secure string parameters. Parameter Store 920 can be used in multiple applications and services subject to policies and permissions provided. When you need to change a parameter value, you change one instance, rather than managing error-prone changes to numerous sources. Secure string parameters are used to manage sensitive data. Parameter Store 902 uses AWS KMS customer master keys (CMKs) to encrypt the parameter values of secure string parameters when it is created or changed. Encryption operations occur on the servers that host EC2 instances, that may execute the security of both data-at-rest and data-in-transit between an instance and its attached EBS storage.

The AWS network architecture provides the facility to select the level of security and resiliency appropriate for the application workload. It helps to build geographically dispersed, fault-tolerant web architectures with cloud resources, AWS has implemented a world-class network infrastructure that is carefully monitored and managed. System 900 may use AWS WAF for establishing the firewall protection to the system.

Layered security is used. Traffic is encrypted, data integrity is authenticated, and the client browser authenticates the identity of the console service endpoint. The system is integrated with third party 910 services to transfer the details such as patient chart, appointments, schedule a virtual visit and to access data from patients' wearable health devices. With the multi-interoperability of communication channels and services, the system may also include an appointment platform may be using a sync model to syncing data which is called a push model, where messages are triggered by user 902. Appointments may be autogenerated based on best results of care, for example monitoring medicine or procedure follow up. In other instances, appointments may be determined by user 902. For e.g. a new appointment is scheduled, a new patient is registered, etc. These messages may be automatically pushed to an established web hook at the system (AWS lambda service). In addition to appointments, orders may be communicated, messages, clinical summaries, and financial transactions, may all be part of the communication and services of System 900.

Generally, AWS lambda 906 acts as a function as a service which receives requests from API Gateway 904. When it is invoked for the first time, AWS Lambda creates an instance of the function and runs its handler method to process the event. When the function returns a response, it stays active and waits to process additional events. If invoked the function again while the first event is being processed, Lambda initializes another instance, and the function processes the two events concurrently. As more events come in, Lambda routes them to available instances and creates new instances as needed. When the number of requests decreases, Lambda stops unused instances to free up scaling capacity for other functions.

Information entering the system may be device acquisition. System 900 may be integrated with a data platform to access the personal health data of a patient from a wearable device. platform and mobile solutions provide continuous access to personal health data using in-home medical devices and wearables. The platform offers a remote patient monitoring solution to administer and support comprehensive virtual care programs. The system may integrate drop in widgets or special areas of data indication. The areas may relate to a specific topic, group of data results, predictions of health, or risk assessment of health care plans and conditions. System 900 may use Natural Language Processing (NLP) to transform a pdf report or a free text into standardized data. System 900 may allow you to process the documents, such as ECG reports. In this embodiment, the server may send events to the stream consumer and to another consumer, which, in turn, submit to either a parser or a data store (including RFMLogs, Vitals, and labs). System 900 looks at vitals, risk, labs and parameters, before sending to a sync with a data store.

In one unlimiting embodiment, the system may focus on a cardiac system 900 or dashboard. The system offers to upload a CMF report, extract the patient details and create the patient record in the database. The CMF report may extract the following details like demographic, allergies, medication, medical and surgical history, family history, smoke and alcoholic details. System 900 offers to upload an Echocardiogram report from a patient chart screen. Following are the entities extracted from an Echo report. Some of the identified data collected with regard to heart function may be as followed, and used in the system to determine cardiac health such as all the individualized extracted heart function data.

System 900 may have two categories of users like the physicians and Nurses who has access to patient chart and the admin users who are responsible for creating native login users for the applications. Each user shall have specific privilege based on the group they are associated with; an application instance will have a site admin, hospital admin, service person, physician, nurses and nurse practitioner. Site admin is created to an instance as a part of initial installation and has all access rights to admin portal, where as a hospital admin will have the rights to create user, associate a user to a group, enable or disable access right.

User 902 (likely a clinician) is created to the system via the methods available in admin portal. Generally, a site admin or a hospital admin can log into an admin portal and create a native login user under the groups available in a customer instance (Physician, Nurse, Nurse practitioner, Hospital Admin or Service Person). Successful user creation shall send an email to the respective user with the initial credentials created by the admin and a link to access system 900. Groups are created to the system as a part of customer instance installation. Each customer instance shall have the following groups.
Physician—Has both read and write permission to Patient Chart and appointments but not to admin interface.
Nurse or Nurse Practitioners—Has read and write permissions to only appointments, vitals and care plan screen. Other screens are provided with only read access.
Hospital Admin—Has both read and write permissions in all the admin modules and do not have access to site level configuration methods.
Site admin—Full access to a particular instance.
Service Person—Has permission to access only the Audit and error logs module.

System 900 may offer communication with patients and clinicians, such as messages may include appointment information, message information clinical summary information, financial information.

For patient health and chart information, profile section deals with patient profile data generated using patient demographics, vitals and procedure reports like ECG, Echo, Cath, Stress and Lab. Below table shows the methods available as a part of patient profile data. In the health state section in the patient chart, the area associated with the information may display the blood works, labs and various physical examination results in a radar chart which is also called as the “Heart Chart”. (See, e.g., FIG. 2, “Heart Score” 202 and the cardio vascular conditions risk predictions 210). System 900 may interact with media, using hardware on the device such as cameras, microphones and speakers for communication (See, e.g., FIG. 7b for an example of telemedia and video communication with a clinician). The information and communication may be analyzed by system 900 for processing during sessions as data input.

In other embodiments of the system, the system may receive patient data related to other patients, different than the patient associated with the dashboard. In this way, the system determines patient(s) that are similar in medical health, physical, or other respects similar to the dashboard patient. Prediction techniques may be applied as described herein to determine similarity and relatedness of the different patient(s). The system will then apply the treatment plans and outcomes of the other patient(s) and use that in the prediction to determine the likelihood of success of treatments as applied to the patient of the dashboard.

The various embodiments described above are provided by way of illustration only and should not be construed to limit the claims attached hereto. Those skilled in the art will readily recognize various modifications and changes that may be made without following the example embodiments and applications illustrated and described herein and without departing from the true spirit and scope of the following claims.

What is claimed is:

1. A system for medical data management for a computing device providing indications for patient care, the system comprising:
a memory;
at least one processor for processing data, wherein the at least one processor:
executes instructions for managing data resources associated with patient care including data extraction and for identifying an element of patient data received;
receives data, based on clinical medical data associated with a patient;
determines a first output associated the received clinical medical data for an output, wherein the first output has at least one area of data associated with the health of the patient;
determines, based on a set of rules of a clinical intelligence engine for providing patient care, a weighted value for each of the respective elements of the received clinical medical data determines, wherein the weighted value is based on a likelihood of a clinician using the data for determining patient care;
determines, based on the weighted value of each of the respective elements of the received clinical medical data, as association with the at least one area of the first output;
selects, based on the determined weighted value of each of the respective elements in relation to a threshold and the association with the at least one area of the first output, the element of the clinical medical data;
determines, based on the determined association of the at least one area, a second output, wherein the at least one of data associated with the health of the patient is included in the second output indicating a suggestion for patient care that the clinician would likely use for patient care; and
outputs, at a graphical user interface, the second output with the likely suggestion for patient care; wherein the second output incorporates the elements associated with clinical medical data, in the at least one area of data, and including the selected element in the second output to a provider taking steps suggested for patient care.

2. The medical data system of claim 1, wherein the data extraction is based on natural language processing.

3. The medical data system of claim 1, wherein the threshold is based on a predetermined value, and when the determined value of the element of the clinical medical data is above the predetermined threshold value, then the element is selected as likely used by the clinician as part of the patient care plan.

4. The medical data system of claim 1, wherein the system identifies natural language in the clinical medical data to determine the second output.

5. The medical data system of claim 1, wherein a clock is applied for the received clinical medical data and for the clinician for accessing the medical data system.

6. The medical data system of claim 1, wherein the clinical medical data comprises, but is not limited to: vital signs, notes, physician input, health, tests, images documents, files, graphical data, and other data associated with the health of the patient.

7. The medical data system of claim 1, wherein the clinician inputs data into the medical data system and is part of the determination of the first output, the second output, or both the first and second outputs.

8. The medical data system of claim 1, wherein the first output may also apply a prediction and clinical intelligence engine to determine the first output

9. The medical data system of claim 1, wherein clinical medical data is at least one of vital signs, notes, clinician input, test results, measurements, documents, photographs, scans, echocardiogram, x-rays, images, and health information associated with the patient.

10. The medical data system of claim 1, wherein the identifying the element is based on discretization, of the element of patient data received.

11. The medical data system of claim 1, wherein determines a value for received a first output based on the received data received from the clinical intelligence engine, which applies rules to the received data at least from the rules engine, and the managed data associated with patient care including care notes, monitoring thresholds and parameters.

12. The medical data system of claim 1, wherein after based on a rules engine of the clinical intelligence engine, for at least one of data verification elements such as the source of information, pattern of information and a likelihood of reliability of data, which a clinician using the data.

13. The medical data system of claim 1, wherein determines a value for received a first output based on the received data received from the clinical intelligence engine, which applies rules to the received data at least from the rules engine, and the managed data associated with patient care including care notes, monitoring thresholds and parameters.

14. The medical data system of claim 1, wherein the managed data associated with patient care includes clinician notes, patient medical monitoring, thresholds, care guidelines, tests, vitals, and parameters.

15. The medical data system of claim 1, wherein the second output includes a graphical display of data representing a visual display of suggested for patient care.

16. The medical data system of claim 1, wherein the executed instruction for managing data resources integrates with the at least one processor and is in response to at least one of receiving data, data management, and a combination thereof.

17. The medical data system of claim 1, wherein the received data from the rules engine includes a verification of data where the likelihood of meeting patient care guidelines and using the data in patient care.

18. The medical data system of claim 1, wherein the executed instruction for managing data resources integrates with the at least one processor for determining if the received data, including at least one of but not limited to health care data, lab result data, testing data, monitoring data, vital sign associated data, medical device data, clinical data, and healthcare data, and the received data is likely associated to the area of the first or second output.

19. The medical data system of claim 1, wherein the processor executes storing data in the memory including, but not limited to, storing patient chart and long records, overall set up information related to patient chart, vital signs, images, pdfs, documents, clinician input, and device data received.

20. The medical data system of claim 1, wherein the processor executes storing data in the memory including, but not limited to, storing selected elements of the received clinical medical data in association with the determined weighted value.

21. The medical data system of claim 1, wherein data determined through the rules engine is determined through a rule or a set of rules in the clinical intelligence platform, and determines an inference or outcome based on the application of the rule or the set of rules.

22. The medical data system of claim 1, wherein the rules engine determines, based on executed instruction for at least managing data resources, the output that includes an integration with medical data associated with a patient chart.

23. The medical data system of claim 1, wherein the determined second output is based on the likelihood of improved or known treatment care guideline for a given patient based on the received clinical intelligence engine data associated with the patient.

24. A method for using a system for medical data management on a computing device providing indications for patient care, the method comprising the steps of:
providing a memory;
providing at least one processor for processing data, wherein the at least one processor:
executes instructions for managing data resources associated with patient care including data extraction and for identifying an element of patient data received;
receives data, based on clinical medical data associated with a patient;
determines a first output associated the received clinical medical data for an output, wherein the first output has at least one area of data associated with the health of the patient;
determines, based on a set of rules of a clinical intelligence engine for providing patient care, a weighted value for each of the respective elements of the received clinical medical data determines, wherein the weighted value is based on a likelihood of a clinician using the data for determining patient care;
determines, based on the weighted value of each of the respective elements of the received clinical medical data, as association with the at least one area of the first output;
selects, based on the determined weighted value of each of the respective elements in relation to a threshold and the association with the at least one area of the first output, the element of the clinical medical data;
determines, based on the determined association of the at least one area, a second output, wherein the at least one of data associated with the health of the patient is included in the second output indicating a suggestion for patient care that the clinician would likely use for patient care; and
outputs, at a graphical user interface, the second output with the likely suggestion for patient care; wherein the second output incorporates the elements associated with clinical medical data, in the at least one area of data, and including the selected element in the second output to a provider taking steps suggested for patient care.
